(19) United States
(12) Plant Patent Application Publication
(10) Pub. No.: US 2008/0235838 P1
(43) Pub. Date: Sep. 25, 2008

(54) Liriope muscari plant named 'LIRF'

(76) Inventor: Todd Anthony Layt, Richmond (AU)

Correspondence Address: Sam R. McCoy Jr., P.O. Box 2108, Mount Pleasant, SC, 29465, US

(21) Appl. No.: 12/077,553

(22) Filed: Mar. 20, 2008

Related U.S. Application Data
(60) Provisional application No. 60/919,717, filed on Mar. 23, 2007.

(51) Int. Cl. A01H 5/00

(52) U.S. Cl. PLT/450

(57) ABSTRACT

‘LIRF’ is a distinctive variety of Liriope muscari which is characterized by the combination of its narrow leaf width, dense foliage and a pink looking flower color.

### LATIN NAME OF THE GENUS AND SPECIES

The Latin name of the genus and species of the novel variety disclosed herein is Liriope muscari.

### VARIETY DENOMINATION

The inventive variety of Liriope disclosed herein has been given the variety denomination ‘LIRF’.

### BACKGROUND OF THE INVENTION

The present invention relates to a new and distinct perennial variety of Liriope muscari, which has been given the variety denomination of ‘LIRF’. Its market class is that of an ornamental plant. ‘LIRF’ is intended for use in landscaping and as a decorative plant.

The Liriope muscari variety ‘LIRF’ was finally selected in 2005 in an Australian nursery in the state of New South Wales following a selection process carried out from 2002 to 2005 involving approximately 20000 seedlings of Liriope muscari cultivar ‘Big Blue’ (unpatented) production stock. ‘LIRF’ is a seedling selection from open pollinated Liriope muscari cultivar ‘Big Blue’. ‘LIRF’ was selected due to its narrow leaf width combined with dense foliage and a pink flower color. ‘LIRF’ was first propagated asexually by division in the state of New South Wales, Australia and has since been asexually propagated by division and micropropagation. The distinctive characteristics of the inventive ‘LIRF’ variety are stable from generation to generation; clones of the variety produced by asexual reproduction maintain the distinguishing characteristics of the original plant.

‘LIRF’ has an upright growth habit with a very dense shoot density whereas ‘Big Blue’ has a semi upright growth habit and a medium shoot density. ‘LIRF’ has a narrow leaf width (mean 5.1 mm in Sydney, NSW Australia) resulting in finer looking foliage whereas ‘Big Blue’ has a broad leaf width (mean 12.1 mm in Sydney, NSW Australia). ‘LIRF’ has an overall pink looking flower color corresponding to violet (RHS 85A) whereas ‘Big Blue’ has a light purple to lilac looking flower color corresponding to violet (RHS 83C).

An application for plant breeders' rights for variety ‘LIRF’ has been lodged with the Australian Plant Breeders' Rights Office, and was first gazetted in 27 March 2006 under Application No. 2006/038.

### SUMMARY OF THE INVENTION

‘LIRF’ is a distinctive variety of Liriope muscari which is characterized by the combination of its narrow leaf width, dense foliage and a pink looking flower color.

### BRIEF DESCRIPTION OF THE FIGURES

FIG. 1 shows a ‘LIRF’ plant.

### BOTANICAL DESCRIPTION OF THE PLANT

The following is a detailed botanical description of a new and distinct variety of a Liriope muscari ornamental plant known as ‘LIRF’. Plant observations were made on plants grown in New South Wales, Australia. Unless indicated otherwise, the descriptions disclosed herein are based upon observations made in February 2006 of mature ‘LIRF’ plants grown in nursery pots.

Those skilled in the art will appreciate that certain characteristics will vary with older or, conversely, younger plants. ‘LIRF’ has not been observed under all possible environmental conditions. Where dimensions, sizes, colors and other characteristics are given, it is to be understood that such characteristics are approximations or averages set forth as accurately as practicable. The phenotype of the variety may vary with variations in the environment such as season, temperature, light intensity, day length, cultural conditions and the like. Color notations are based on The Royal Horticultural Society Colour Chart, The Royal Horticultural Society, London, 1995 edition. Note that generic color descriptions such as ‘pink’ do not exist in the RHS charts and the corresponding RHS colors are quoted.

‘LIRF’ is a perennial Liriope muscari plant which is a seedling selection from a Liriope muscari cultivar ‘Big Blue’. After its selection, ‘LIRF’ was asexually propagated by division and then by micropropagation. ‘LIRF’ has a green leaf color and pink flowers displayed within the foliage.

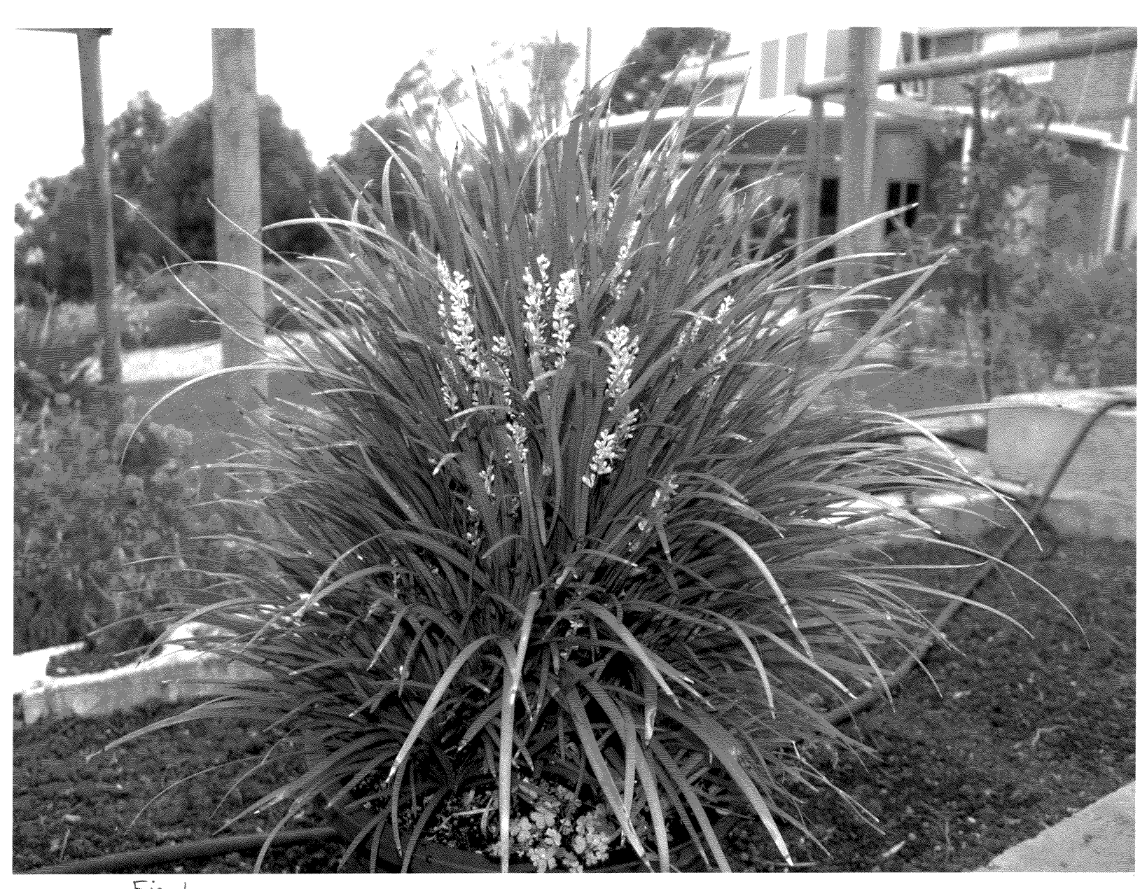

These features and other characteristics are apparent from the description provided below.
Growth habit, dimensions and color: ‘LIRF’ is a short-medium plant forming an upright clump with slightly spreading leaves. Average plant height is to 50-55 cm and average plant spread is 65-85 cm in a mature plant grown in a garden in Sydney, New South Wales, Australia (observed January 2007). A representative ‘LIRF’ plant is shown in FIG. 1. The upper side of the mature leaf is yellow green in color (observed January 2007) corresponding to approximately RHS 147A. Leaf margins and mid ribs do not contrast prominently with the leaf blade. The leaf lower side color corresponds to the upper leaf side but has a less glossy surface texture. The leaf is narrow in width (mean 5.1 mm in Sydney, NSW Australia), and reaches an average length of 27 cm. The leaf shape is ligulate-ensiform, leaf base is cauline, leaf margin is entire, leaf apex is acute and leaf surface texture is glossy and glabrous. The leaf venation pattern is parallel; the color is the same as the rest of the leaf. The leaf attitude is erect to semi erect with maturity.
Roots: The roots of ‘LIRF’ are colored white (approximately RHS 155C) and fleshy and thick, similar to other Liriope muscari.
Inflorescence: The inflorescence is a raceme with flowers borne in clusters of typically 4 or 5 and reducing in number near the apex. Each flower cluster consists of pedicellate flowers ranging from very a short pedicel length (almost sessile; less than 1 mm) to up to 3.5 mm long. Inflorescence length is approximately 24 cm on average and the raceme position is within the foliage. Raceme length is 50-60 mm typically. Peduncle color is yellow green approximately RHS 148A distally and changing to brown (RHS 200C) at the base. Each flower cluster is subtended by a small bract colored yellow green approximately RHS 146B. Pedicel color is purple RHS 78D. The flowering season in Sydney, NSW, Australia is late December-January until late February.
Flowers: Flower bud color is violet approximately RHS 84D (immature) to RHS 86D (mature). Mature petal color is violet RHS 85A. Anther color is yellow RHS 7C and stigma color is purple violet RHS 82D. Flower diameter range is 8 mm-11 mm when fully opened.
Comparison of lirf with other varieties of liriope muscari: ‘LIRF’ has a narrower leaf width (mean 5.1 mm) and very dense foliage compared to common Liriope muscari which has a medium leaf width (mean 8 mm) and medium density foliage. ‘LIRF’ has a very narrow leaf width (mean 5.1 mm) and very dense foliage whereas ‘Big Blue’ has a broad leaf width (mean 12.1 mm) and medium density foliage. ‘LIRF’ has a medium plant height (50-55 cm) with medium leaf length (mean 27 cm) whereas ‘Big Blue’ has a tall plant height (to 75 cm) with long leaf length (mean 30 cm). ‘LIRF’ has a ‘pink’ looking flower color corresponding to violet (RHS 85A) whereas ‘Big Blue’ has a flower color corresponding to violet (RHS 83C). ‘LIRF’ differs from the variety ‘Bigun’ (U.S. Plant Pat. No. 15,474) as it has a shorter plant height (50-55 cm), narrower leaf width (mean 5.1 mm), an absence of leaf twisting and pink flowers corresponding to violet (RHS 85A) whereas ‘Bigun’ has a medium plant height (to 60 cm), broad leaf width (to 18 mm), leaf twisting present and dark violet colored flowers. ‘LIRF’ differs from the variety ‘Samantha’ (unpatented) as it has a more upright plant growth habit, a narrower leaf width (mean 5.1 mm), and deeper pink flowers corresponding to violet (RHS 85A) whereas ‘Samantha’ has a more semi-upright plant growth habit, a medium-broad leaf width (mean 9.3 mm) and lighter pink colored flowers corresponding to purple (RHS 78C). The combination of its dense upright growth habit, very narrow leaf width and pink flower color makes ‘LIRF’ a desirable ornamental plant suited for mass production for pot and landscape use.
Asexual reproduction: After its initial discovery, ‘LIRF’ was transplanted into a 140 mm pot for further trials and testing. After divisions were made for several subsequent generations, ‘LIRF’ was observed to retain color and size characteristics that were noted in the original ‘LIRF’ seedling. Divisions of ‘LIRF’ root within 2 to 3 weeks. ‘LIRF’ was divided into several pots for further evaluation. Potting mix used contained 50% sand and 50% peat. Plants were divided into 90 mm×50 mm×50 mm tubes. ‘LIRF’ has been propagated in vitro using micropropagation and has been observed to reproduce from generation to generation in a uniform and stable manner maintaining original color and size characteristics.
Environmental tolerances: ‘LIRF’ has typical shade tolerance for the species and performed the same as the variety ‘Evergreen Giant’ in garden trials at Clarendon, NSW, Australia. The winter hardiness of ‘LIRF’ is at least to zone 5A in the United States, and evaluation of winter hardiness is ongoing. ‘LIRF’ has drought tolerance similar to the variety ‘Evergreen Giant’ with moderate to good recovery with watering after severe wilting. ‘LIRF’ survived and grew well over 2 years in garden trials in sandy and clay soils, but also tolerates heavy, clay-type soils at Clarendon, NSW, Australia.
Disease resistance: ‘LIRF’ has typical resistance to fungal and bacterial pathogens compared to other similar Liriope muscari cultivars as demonstrated in garden trials with survival and growth vigor similar to the variety ‘Evergreen Giant’ at Clarendon, NSW, Australia.

That which is claimed is:

1. A new and distinct variety of Liriope muscari plant named ‘LIRF’, substantially as described and illustrated herein.
